(19) United States
(12) Patent Application Publication
(10) Pub. No.: US 2026/0242739 A1
(43) Pub. Date: Aug. 20, 2026

(54) ROLE OF HEPATOCYTE-DERIVED EXTRACELLULAR MATRIX AND COAGULATION FACTORS FOR THE FATE RESTORATION AND MAINTENANCE OF HUMAN HEPATOCYTES

(71) Applicants: UNIVERSITY OF SOUTHERN CALIFORNIA, Los Angeles, CA (US); PhoenixBio Co., Ltd., Higashi-Hiroshima-shi, Hiroshima (JP)

(72) Inventors: Takeshi SAITO, Los Angeles, CA (US); Yuji ISHIDA, Hiroshima (JP); Go SUGAHARA, Hiroshima (JP)

(73) Assignees: UNIVERSITY OF SOUTHERN CALIFORNIA, Los Angeles, CA (US); PhoenixBio Co., Ltd., Higashi-Hiroshima-shi, Hiroshima (JP)

(21) Appl. No.: 19/160,603

(22) PCT Filed: Mar. 4, 2024
(86) PCT No.: PCT/JP2024/080021

§ 371 Date: Aug. 28, 2025

Related U.S. Application Data
(60) Provisional application No. 63/449,401, filed on Mar. 2, 2023.

(51) Int. Cl. C12N 5/071

(52) U.S. Cl. CPC C12N 5/067; C12N 2500/30; C12N 2500/32; C12N 2500/38; C12N 2501/11; C12N 2501/33; C12N 2501/72; C12N 2501/998; C12N 2501/999

(57) ABSTRACT

Disclosed herein are human hepatocyte culture media and methods that better support the fate restoration and maintenance of cultured human hepatocytes. Culturing human hepatocytes is improved with the supplementation of DMSO or DMSO2-supplemented hepatocyte clonal growth media or hepatocyte maintenance media with any one or a combination of the factors of fibronectin, laminin, vitronectin, serpin, and coagulation factors.

### FIELD

The present disclosure relates to cell culture medium and uses thereof.

### BACKGROUND

Chronic liver diseases (CLD), such as viral hepatitis (HBV and HCV), alcoholic liver disease (ALD), non-alcoholic fatty liver disease (NAFLD), as well as numerous genetic diseases of the liver leads to the development of end-stage liver diseases (ESLD) such as decompensated liver failure and liver cancer. Currently, ESLD is the 11^{th} leading cause of death worldwide, and the only definitive medical intervention is liver transplantation (LT). However, LT is an invasive procedure, and the nationwide organ shortage greatly limits its utility. In addition, LT recipients are required to receive lifelong immunosuppressive therapy, which is associated with significant morbidity, including the development of opportunistic infections and malignant neoplasms.

Hepatocyte transplantation technology is anticipated to eventually replace LT; however, various obstacles remain, the most significant of which is the lack of a definite technology that permits the complete maturation of iPS cell-derived human hepatocytes, referred to as iHep. The degree of the maturation of currently available iHeps are still far below compared to that of primary human hepatocytes and at most at the level of bipotential hepatic progenitor cells. Accordingly, iHeps is incapable of proliferate in the host mouse of humanized liver chimeric mice, which is in stark contrast to the characteristic of mature hepatocytes. Thus, there remains a substantial unmet need for technology that facilitates the definitive maturation of hepatic progenitor cells.

In addition, it is important to note that the successful establishment of hepatocyte transplantation therapy requires furthering the understanding of the molecular/cellular biology of the liver. One of the current fundamental limitations is the lack of a definitive technology that allows the cell fate maintenance of matured human hepatocytes in vitro.

The findings disclosed in the following sections are critical assets for the regenerative/stem cell biology field towards the establishment of hepatocyte transplantation therapy. Furthermore, the disclosed invention provides a substantial degree of benefit for the basic research of liver biology and disorders.

### SUMMARY

The present invention provides as follows.
[1]A hepatocyte culture medium supplemented with at least one factor selected from the group consisting of: Fibronectin (FN), laminin (LAM), vitronectin (VTN), orosomucoid 1 (ORM1), serpin, collagen, haptoglobin (HP), transglutaminase 2 (TGM2), alpha-2 macroglobulin (A2M), coagulation factors including but not limited to fibrinogen (FG), Tenascin C (TNC) and combinations thereof.
[2] The culture medium of [1], wherein the medium is a hepatocyte clonal growth medium (HCGM) or hepatocyte maintenance medium (HMM).
[3] The culture medium of [1], wherein the laminin is laminin subunit gamma 1 (LAMC1), laminin subunit gamma 2 (LAMC2), or laminin subunit gamma 3 (LAMC3).
[4] The culture medium of [1], wherein the coagulation factor is fibrinogen alpha chain (FGA), fibrinogen beta chain (FGB), or fibrinogen gamma chain (FGG).
[5] The culture medium of any one of [1]-[4], wherein the medium is further supplemented with DMSO or DMSO2.
[6] The culture medium of any one of [2]-[5], wherein the HCGM comprises, consists, or consists essentially of, Dulbecco's Modified Eagle's Medium (DMEM), L-proline, insulin, dexamethasone, EGF, and L-ascorbic acid 2-phosphate (Asc-2P).
[7] The culture medium of [6], wherein the DMEM is DMEM-10, and the DMEM-10 comprises, consists, or consists essentially of, DMEM, HEPES, penicillin-streptomycin, and fetal bovine serum (FBS).
[8] The culture medium of [6], wherein the L-proline is 15 μg/mL in the HCGM, the insulin is 0.25 μg/mL in the HCGM, the dexamethasone is 50 nM in the HCGM, the EGF is 5 ng/mL in the HCGM, the Asc-2P is 0.1 mM in the HCGM, the DMSO or the DMSO2 is 2% or 140.8 mM respectively in the HCGM, the HEPES is 20 mM in the HCGM, the penicillin is 100 IU/mL in the HCGM, the streptomycin is 100 μg/mL in the HCGM, and the FBS is heat-inactivated FBS at 10% in the HCGM.
[9] The culture medium [6], wherein the L-proline is 5-25 μg/mL in the HCGM, the insulin is 0.1-0.5 μg/mL in the HCGM, the dexamethasone is 10-100 nM in the HCGM, the EGF is 1-10 ng/mL in the HCGM, the Asc-2P is 0.01-1 mM in the HCGM, the DMSO or the DMSO2 is 0.5%-5% 100-180 mM, respectively in the HCGM, the HEPES is 10-50 mM in the HCGM, the penicillin is 10-300 IU/mL in the HCGM, the streptomycin is 10-300 μg/mL in the HCGM, and the FBS is heat-inactivated FBS at 2-20% in the HCGM.
[10] The culture medium of any one of [2]-[5], wherein the HMM comprises, consists, or consists essentially of, Dulbecco's Modified Eagle's Medium (DMEM), L-proline, insulin, dexamethasone, and L-ascorbic acid 2-phosphate (Asc-2P).
[11] The culture medium of [10], wherein the DMEM is DMEM-10, and the DMEM-10 comprises, consists, or consists essentially of, DMEM, HEPES, penicillin-streptomycin, and fetal bovine serum (FBS).
[12] The culture medium of [10], wherein the L-proline is 15 μg/mL in the HMM, the insulin is 0.25 μg/mL in the HMM, the dexamethasone is 50 nM in the HMM, the Asc-2P is 0.1 mM in the HMM, the DMSO or the DMSO2 is 2% or 140.8 mM respectively in the HMM, the HEPES is 20 mM in the HMM, the penicillin is 100 IU/mL in the HMM, the streptomycin is 100 μg/mL in the HMM, and the FBS is heat-inactivated FBS at 10% in the HMM.
[13] The culture medium of [10], wherein the L-proline is 5-25 μg/mL in the HMM, the insulin is 0.1-0.5 μg/mL in the HMM, the dexamethasone is 10-100 nM in the HMM, the Asc-2P is 0.01-1 mM in the HMM, the DMSO or the DMSO2 is 0.5%-5% or 100-180 mM, respectively in the HMM, the HEPES is 10-50 mM in the HMM, the penicillin is 10-300 IU/mL in the HMM, the streptomycin is 10-300 μg/mL in the HMM, and the FBS is heat-inactivated FBS at 2-20% in the HMM.
[14] The culture medium of any one of [2]-[13], wherein the FN is supplemented to 10000 ng/mL in the HCGM or HMM.
[15] The culture medium of any one of [2]-[13], wherein the laminin is supplemented to 1-10000 μg/mL in the HCGM or HMM.
[16] The culture medium of any one of [2]-[13], wherein the VTN is supplemented to 0.1-1000 ng/mL in the HCGM or HMM.
[17] The culture medium of any one of [2]-[13], wherein the ORM1 is supplemented to 1-10000 μg/mL in the HCGM or HMM.
[18] The culture medium of any one of [2]-[13], wherein the serpin is supplemented to 0.1-1000 μg/mL in the HCGM or HMM.
[19] The culture medium of any one of [2]-[13], wherein the collagen is supplemented to 1-10000 ng/mL in the HCGM or HMM.
[20] The culture medium of any one of [2]-[13], wherein the HP is supplemented to 0.01-100 mg/mL in the HCGM or HMM.
[21] The culture medium of any one of [2]-[13], wherein the TGM2 is supplemented to 0.01-100 ng/mL in the HCGM or HMM.
[22] The culture medium of any one of [2]-[13], wherein the A2M is supplemented to 0.1-1000 μg/mL in the HCGM or HMM.
[23] The culture medium of any one of [2]-[13], wherein the coagulation factor is supplemented to 0.1-1000 mg/mL in the HCGM or HMM.
[24] The culture medium of any one of [2]-[13], wherein the TNC is supplemented to 0.01-100 ng/mL in the HCGM or HMM.
[25] A culture medium of any one of [1]-[24] for use in in cultivating terminally differentiated human hepatocytes, hepatocytes derived from a chimeric animal with humanized liver, and/or cryopreserved and/or freshly isolated primary human hepatocytes.
[26] A culture medium of any one of [1]-[24] for use in differentiating hepatic bipotential progenitor cells (iHep) and/or chemically induced liver progenitors into terminally differentiated hepatocytes or matured hepatocytes, respectively.
[27] A culture medium of any one of [1]-[24] for use in co-culturing with nonparenchymal cells, and/or for use in suspending human hepatocytes in cryopreservation.
[28] A culture medium of any one of [1]-[24] for use in the expansion and/or propagation of iHep and Chemically Induced Liver Progenitors (CLiPs) in the liver of HLCM-host mouse.
[29] A method of cultivating terminally differentiated human hepatocytes, hepatocytes derived from a chimeric animal with humanized liver, and/or cryopreserved and/or freshly isolated primary human hepatocytes, the method comprising culturing a quantity of hepatocytes in a cell culture medium of any one of [1]-[24].
[30] A method of differentiating hepatic bipotential progenitor cells (iHep) and/or chemically induced liver progenitors into terminally differentiated hepatocytes or matured hepatocytes, respectively, the method comprising culturing a quantity of hepatocytes in a cell culture medium of any one of [1]-[24].
[31] A method of co-culturing human hepatocytes with nonparenchymal cells, and/or suspending human hepatocytes in cryopreservation, the method comprising culturing a quantity of hepatocytes in a cell culture medium of any one of [1]-[24].
[32] A method of expanding and/or propagating iHep and CLiPs in the liver of HLCM-host mouse, the method comprising culturing a quantity of hepatocytes in a cell culture medium of any one of [1]-[24].

### BRIEF DESCRIPTION OF THE DRAWINGS

FIG. 1

Detection of HLCM-HH derived humoral factors in the CM. The CM was collected as described in materials and methods and examined by immunoblotting. All signals were detected at expected molecular weight.

FIG. 2.

Results of gene expression analysis of HLCM-HHs cultured with dHCGM in the presence or absence of fibrinogen. HLCM-HHs were plated on collagen-coated 24-w plates and cultured with dHCGM or fibrinogen (50 μg/mL) supplemented dHCGM for 7 days. Y-axis title is indicated on the top left panel. Bars represents average ±S.D.*: p<0.05, **: p<0.01, ***: p<0.001, #: p<0.0001.

FIG. 3.

Results of gene expression analysis of HLCM-HHs cultured with dHCGM, CM, and CM containing coagulation cascade inhibitors. HLCM-HHs were plated on collagen-coated 24-w plates and cultured with dHCGM, CM, or CM containing F2i (Dabigatran etexilate, 2 μg/mL) or F10i (Rivoroxaban, 400 ng/mL) for 7 days. Y-axis title is indicated on the top left panel. Bars represents average ±S.D. #: p<0.05, vs FM, FM+F2i, and FM+F10i, *: p<0.05, **: p<0.01, ***: p<0.001, ****: p<0.0001.

FIG. 4.

Results of gene expression analysis of HLCM-HHs cultured by commercially available hepatocyte culture media supplemented with or without fibrinogen. HLCM-HHs were plated on collagen-coated 24-w plates and cultured by IH or HCM in the presence or absence of fibrinogen (50 μg/mL) for 7 days. Y-axis title is indicated on the top left panel. Bars represents average ±S.D.****: p<0.0001.

FIG. 5.

Results of gene expression analysis of HLCM-HHs cultured with dHCGM containing CM component(s). HLCM-HHs were plated on collagen-coated 24-w plates and cultured by dHCGM, CM, dHCGM supplemented with one or combination of the following CM components for 7 days; VTN: 50 ng/mL, FN: 100 ng/mL, TNC: 2 ng/mL, HP: 0.03 U/mL. Y-axis title is indicated on the top left panel. Bars represents average ±S.D.*: p<0.05, ?: p<0.01, ***: p<0.001, #: p<0.0001.

FIG. 6.

Results of gene expression analysis of HLCM-HHs transfected with A2M specific siRNA. HLCM-HHs were plated on siRNA transfection plates coated with negative control or A2M specific siRNA, and cultured with dHCGM for 7 days. Y-axis represents relative expression level to freshly isolated HLCM-HHs. Bars represents average ±S.D.***: p<0.001.

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed, Cold Spring Harbor Laboratory Press (Cold Spring Harbor, NY 2012), provide one skilled in the art with a general guide to many of the terms used in the present application. One skilled in the art will recognize many methods and materials similar or equivalent to those described herein, which could be used in the practice of the present invention. Indeed, the present invention is in no way limited to the methods and materials described. For purposes of the present invention, several terms are defined below.

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.” and “for example” set off lists of one or more non-limiting examples, instances, or illustrations.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The components, steps, features, objects, benefits and advantages which have been discussed are merely illustrative. None of them, nor the discussions relating to them, are intended to limit the scope of protection in any way. Numerous other embodiments are also contemplated. These include embodiments which have fewer, additional, and/or different components, steps, features, objects, benefits and advantages. These also include embodiments in which the components and/or steps are arranged and/or ordered differently.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

All articles, patents, patent applications, and other publications that have been cited in this disclosure are incorporated herein by reference.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician. Relational terms such as “first” and “second” and the like may be used solely to distinguish one entity or action from another, without necessarily requiring or implying any actual relationship or order between them.

One skilled in the art will recognize many methods and materials similar or equivalent to those described herein, which could be used in the practice of the present invention. Indeed, the present invention is in no way limited to the methods and materials described. For purposes of the present invention, several terms are defined below.

The Applicants' invention can be used for the regenerative/stem cell biology field towards the establishment of hepatocyte transplantation therapy. Furthermore, the invention also provides a substantial degree of benefit for the basic research of liver biology and pathophysiology in general.

The Applicants found that there are several key hepatocyte-intrinsic humoral factors contained the culture medium of terminally differentiated human hepatocytes (HH) (hereafter conditioned medium of human hepatocytes; CMHH). These factors can be used individually or in combination as supplements to DMSO or DMSO2 containing hepatocyte clonal growth medium or DMSO or DMSO2 containing hepatocyte maintenance medium (Tables 1-3).

As used herein, in various aspects, combination of factors is a combination of two, or a combination of three, or a combination of four . . . etc. These humoral factors are fibronectin (FN), e.g., FN1; laminin, e.g., LAMC1, LAMC2 and LAMC3; vitronectin (VTN); Orosomucoid 1 (ORM1) or Alpha-1-Acid Glycoprotein, (AGP1); serpin, e.g., SERPINA3; collagen such as type 4 collagen, e.g., collagen IV Alpha 1 Chain; Haptoglobin (HP); Transglutaminase 2 (TGM2); alpha-2 macroglobulin (A2M); Tenascin C (TNC), and coagulation factors such as fibrinogen, e.g., FGA, FGB, and FGG). The humoral factor supplemented media provide advantages for use in regenerative medicine and stem cell biology, e.g., to facilitate cell terminal maturation of iHeps and cell fate maintenance of fully matured iHeps. In addition, supplemented media provides support for liver cell biology research, e.g., supporting the cell fate maintenance, function, and longevity of in vitro cultured HH, primary hepatocytes from other species (e.g., dog, murine rat, and monkey), hepatocytes derived from humanized liver chimeric animals, and nonparenchymal cells (NPC) of the liver.

In some embodiments, the medium being supplemented with one or a combination of FN, laminin, VTN, ORM1, serpin, collagen, HP, TGM2, A2M, TNC, and/or coagulation factor is a hepatocyte clonal growth medium containing DMSO (dHCGM), as shown in Tables 1 and 3. In some embodiments, dHCGM comprises, or consists essentially of, a standard cell culture base medium, L-proline, insulin, dexamethasone, EGF, and L-ascorbic acid 2-phosphate (Asc-2P). Particularly desired, the standard cell culture base medium is supplemented with HEPES, penicillin-streptomycin, and serum. In some embodiments the serum is fetal bovine serum (FBS) or human serum. In preferred embodiments, the standard cell culture base medium is Dulbecco's Modified Eagle's Medium (DMEM)-10.

Standard cell culture base media are well known to those skilled in the art. Examples of standard cell culture base media include but are not limited to DMEM, DMEM-10, Minimum Essential Media (MEM), RPMI-1640, Iscove's Modified Dulbecco's Medium (IMDM), or William's E Medium

In some embodiments, the medium being supplemented with one or a combination of FN, laminin, VTN, ORM1, serpin, collagen, HP, TGM2, A2M, TNC, and/or coagulation factor is a DMSO-supplemented hepatocyte maintenance medium (dHMM), as shown in Tables 2 and 3. In some embodiments dHMM comprises, or consists essentially of, DMSO, a standard cell culture base medium, L-proline, insulin, dexamethasone, and L-ascorbic acid 2-phosphate (Asc-2P) Particularly desired, the standard cell culture base medium is supplemented with HEPES, penicillin-streptomycin, and serum. In some embodiments the serum is fetal bovine serum (FBS) or human serum. In preferred embodiments, the standard cell culture base medium is Dulbecco's Modified Eagle's Medium (DMEM)-10.

In some embodiments, the medium being supplemented with one or a combination of FN, laminin, VTN, ORM1, serpin, collagen, HP, TGM2, A2M, TNC, and/or coagulation factor is DMSO2-supplemented hepatocyte clonal growth medium (d2HCGM), rather than DMSO-supplemented, as shown in Tables 2 and 3. In some embodiments, the second HH culture medium is a tetramethylene sulfoxide (TMSO)-supplemented hepatocyte clonal growth medium (tHCGM), rather than DMSO-supplemented.

In some embodiments, the medium being supplemented with one or a combination of FN, laminin, VTN, ORM1, serpin, collagen, HP, TGM2, A2M, TNC, and/or coagulation factor, e.g., d2HCGM, comprises or consists essentially of DMSO2, a standard cell culture base medium, L-proline, insulin, dexamethasone, EGF, and Asc-2P. Particularly desired, the standard cell culture base medium is supplemented with HEPES, penicillin-streptomycin, and serum. In some embodiments the serum is fetal bovine serum (FBS) or human serum. In preferred embodiments, the standard cell culture base medium is Dulbecco's Modified Eagle's Medium (DMEM)-10.

In some embodiments, the medium being supplemented with one or a combination of FN, laminin, VTN, ORM1, serpin, collagen, HP, TGM2, A2M, TNC, and/or coagulation factor, e.g., tHCGM, comprises or consists essentially of TMSO, a standard cell culture base medium, L-proline, insulin, dexamethasone, EGF, and Asc-2P. Particularly desired, the standard cell culture base medium is supplemented with HEPES, penicillin-streptomycin, and serum. In some embodiments the serum is fetal bovine serum (FBS) or human serum. In preferred embodiments, the standard cell culture base medium is Dulbecco's Modified Eagle's Medium (DMEM)-10.

In some embodiments, the medium being supplemented with one or a combination of FN, laminin, VTN, ORM1, serpin, collagen, HP, TGM2, A2M, TNC, and/or coagulation factor is DMSO2-supplemented hepatocyte maintenance medium (d2HMM), rather than DMSO-supplemented, as shown in Tables 2 and 3. In some embodiments, the second HH culture medium is a tetramethylene sulfoxide (TMSO)-supplemented hepatocyte maintenance medium (tHMM), rather than DMSO-supplemented.

In some embodiments, the medium being supplemented with one or a combination of FN, laminin, VTN, ORM1, serpin, collagen, HP, TGM2, A2M, TNC, and/or coagulation factor, e.g., d2HMM, comprises or consists essentially of DMSO2, a standard cell culture base medium, L-proline, insulin, dexamethasone, and Asc-2P. Particularly desired, the standard cell culture base medium is supplemented with HEPES, penicillin-streptomycin, and serum. In some embodiments the serum is fetal bovine serum (FBS) or human serum. In preferred embodiments, the standard cell culture base medium is Dulbecco's Modified Eagle's Medium (DMEM)-10.

In some embodiments, the medium being supplemented with one or a combination of FN, laminin, VTN, ORM1, serpin, collagen, HP, TGM2, A2M, TNC, and/or coagulation factor, e.g., tHMM, comprises or consists essentially of TMSO, a standard cell culture base medium, L-proline, insulin, dexamethasone, and Asc-2P. Particularly desired, the standard cell culture base medium is supplemented with HEPES, penicillin-streptomycin, and serum. In some embodiments the serum is fetal bovine serum (FBS) or human serum. In preferred embodiments, the standard cell culture base medium is Dulbecco's Modified Eagle's Medium (DMEM)-10.

In some embodiments, the medium being supplemented with one or a combination of FN, laminin, VTN, ORM1, serpin, collagen, HP, TGM2, A2M, TNC, and/or coagulation factor, e.g., dHCGM, d2HCGM, or tHCGM, is supplemented with an amount of DMSO, DMSO2, or TMSO that is at least 70 mM, 35 mM, and 35 mM, respectively in a standard cell culture base medium, wherein the standard cell culture base medium further includes one or more of L-proline, insulin, dexamethasone, EGF, Asc-2P, HEPES, penicillin-streptomycin, and serum. In some embodiments the serum is FBS or human serum. In preferred aspects, the standard cell culture base medium is DMEM-10.

In some embodiments, the medium being supplemented with one or a combination of FN, laminin, VTN, ORM1, serpin, collagen, HP, TGM2, A2M, TNC, and/or coagulation factor, e.g., dHCGM, d2HCGM, or tHCGM, is supplemented with an amount of DMSO, DMSO2, or TMSO to reach a concentration of about 281.6 mM, 140.8 mM, and 70.4 mM, respectively in a standard cell culture base medium, wherein the standard cell culture base medium further includes one or more of L-proline, insulin, dexamethasone, EGF, Asc-2P, HEPES, penicillin-streptomycin, and serum. In some embodiments the serum is FBS or human serum. In preferred aspects, the standard cell culture base medium is DMEM-10.

In some embodiments, the medium being supplemented with one or a combination of FN, laminin, VTN, ORM1, serpin, collagen, HP, TGM2, A2M, TNC, and/or coagulation factor, e.g., dHMM, d2HMM, or tHMM, is supplemented with an amount of DMSO, DMSO2, or TMSO that is at least 70 mM, 35 mM, and 35 mM, respectively in a standard cell culture base medium, wherein the standard cell culture base medium further includes one or more of L-proline, insulin, dexamethasone, Asc-2P, HEPES, penicillin-streptomycin, and serum. In some embodiments the serum is FBS or human serum. In preferred aspects, the standard cell culture base medium is DMEM-10.

In some embodiments, the medium being supplemented with one or a combination of FN, laminin, VTN, ORM1, serpin, collagen, HP, TGM2, A2M, TNC, and/or coagulation factor, e.g., dHMM, d2HMM, or tHMM, is supplemented with an amount of DMSO, DMSO2, or TMSO to reach a concentration of about 281.6 mM, 140.8 mM, and 70.4 mM, respectively in a standard cell culture base medium, wherein the standard cell culture base medium further includes one or more of L-proline, insulin, dexamethasone, Asc-2P, HEPES, penicillin-streptomycin, and serum. In some embodiments the serum is FBS or human serum. In preferred aspects, the standard cell culture base medium is DMEM-10.

In embodiments, the medium being supplemented with one or a combination of FN, laminin, VTN, ORM1, serpin, collagen, HP, TGM2, A2M, TNC, and/or coagulation factor, e.g., dHCGM, d2HCGM, or tHCGM, is supplemented with an amount of DMSO, DMSO2, or TMSO is supplemented to reach a concentration of about 2 (v/v) % in a standard cell culture base medium, wherein the standard cell culture base medium further includes one or more of L-proline, insulin, dexamethasone, EGF, Asc-2P, HEPES, penicillin-streptomycin, and serum. In some embodiments the serum is FBS or human serum. In preferred aspects, the standard cell culture base medium is DMEM-10.

In embodiments, the medium being supplemented with one or a combination of FN, laminin, VTN, ORM1, serpin, collagen, HP, TGM2, A2M, TNC, and/or coagulation factor, e.g., dHMM, d2HMM, or tHMM, is supplemented with an amount of DMSO, DMSO2, or TMSO is supplemented to reach a concentration of about 2 (v/v) % in a standard cell culture base medium, wherein the standard cell culture base medium further includes one or more of L-proline, insulin, dexamethasone, Asc-2P, HEPES, penicillin-streptomycin, and serum. In some embodiments the serum is FBS or human serum. In preferred aspects, the standard cell culture base medium is DMEM-10.

In some embodiments, the medium being supplemented with one or a combination of FN, laminin, VTN, ORM1, serpin, collagen, HP, TGM2, A2M, TNC, and/or coagulation factor comprises the DMSO at 281.6 mM or 2% (v/v), and a standard cell culture base medium, wherein the medium comprises one or more of L-proline at 15 μg/mL, insulin at 0.25 μg/mL, dexamethasone at 50 nM, EGF at 5 ng/mL, Asc-2P at 0.1 mM, HEPES at 20 mM, penicillin at 100 IU/mL, streptomycin at 100 μg/mL, and the -inactivated FBS or human serum at 10%. In preferred aspects, the standard cell culture base medium is DMEM-10.

In some embodiments, the medium being supplemented with one or a combination of FN, laminin, VTN, ORM1, serpin, collagen, HP, TGM2, A2M, TNC, and/or coagulation factor comprises the DMSO2 or TMSO at 140.8 mM or 70.4 mM, respectively, and a standard cell culture base medium, wherein the medium comprises one of more of L-proline at 15 μg/mL, insulin at 0.25 μg/mL, dexamethasone at 50 nM, EGF at 5 ng/mL, Asc-2P at 0.1 mM, HEPES at 20 mM, penicillin at 100 IU/mL, streptomycin at 100 μg/mL, and the heat-inactivated FBS or human serum at 10%. In preferred aspects, the standard cell culture base medium is DMEM-10.

In some embodiments, the medium being supplemented with one or a combination of FN, laminin, VTN, ORM1, serpin, collagen, HP, TGM2, A2M, TNC, and/or coagulation factor comprises the DMSO at 0.5%-5% (v/v) and the a standard cell culture base medium, wherein the medium comprises one or one of the L-proline at 5-25 μg/mL, the insulin at 0.1-0.5 μg/mL, the dexamethasone at 10-100 nM, the EGF at 0-10 ng/mL, the Asc-2P at 0.01-1 mM, the HEPES at 10-50 mM, the penicillin at 10-300 IU/mL, the streptomycin at 10-300 μg/mL, and the heat-inactivated FBS or human serum at 2-20%. In preferred aspects, the standard cell culture base medium is DMEM-10.

In some embodiments, the medium being supplemented with one or a combination of FN, laminin, VTN, ORM1, serpin, collagen, HP, TGM2, A2M, TNC, and/or coagulation factor comprises the DMSO, DMSO2, or TMSO at 35-281.6 mM and the a standard cell culture base medium, wherein the medium comprises one or one of the L-proline at 5-25 μg/mL, the insulin at 0.1-0.5 μg/mL, the dexamethasone at 10-100 nM, the EGF at 0-10 ng/mL, the Asc-2P at 0.01-1 mM, the HEPES at 10-50 mM, the penicillin at 10-300 IU/mL, the streptomycin at 10-300 μg/mL, and the heat-inactivated FBS or human serum at 2-20%. In preferred aspects, the standard cell culture base medium is DMEM-10.

In some embodiments the medium being supplemented with one or a combination of FN, laminin, VTN, ORM1, serpin, collagen, HP, TGM2, A2M, TNC, and/or coagulation factor comprises DMSO at a concentration of 150 mM-350 mM, at a concentration of 200 mM-300 mM, at a concentration of 220 mM-290 mM, at a concentration of 240 mM-285 mM, at a concentration of 281.6 mM.

In some embodiments the medium being supplemented with one or a combination FN, laminin, VTN, ORM1, serpin, collagen, HP, TGM2, A2M, TNC, and/or coagulation factor comprises DMSO2 at a concentration of 100 mM-200 mM, at a concentration of 120 mM-180 mM, at a concentration of 140 mM-160 mM, at a concentration of 140.8 mM.

In some embodiments the medium being supplemented with one or a combination of FN, laminin, VTN, ORM1, serpin, collagen, HP, TGM2, A2M, TNC, and/or coagulation factor comprises TMSO at a concentration of 40 mM-100 mM, at a concentration of 50 mM-90 mM, at a concentration of 60 mM-80 mM, at a concentration of 70.4 mM.

In some embodiments the medium being supplemented with one or a combination of FN, laminin, VTN, ORM1, serpin, collagen, HP, TGM2, A2M, TNC, and/or coagulation factor comprises L-proline at a concentration of 5-25 μg/mL, at a concentration of 10-20 μg/mL, at a concentration of 12-18 μg/mL, at a concentration of 15 μg/mL.

In some embodiments the medium being supplemented with one or a combination of FN, laminin, VTN, ORM1, serpin, collagen, HP, TGM2, A2M, TNC, and/or coagulation factor comprises insulin at a concentration of 0.1-0.5 μg/mL, at a concentration of 0.15-0.45 μg/mL, at a concentration of 0.2-0.4 μg/mL, at a concentration of 0.25 μg/mL.

In some embodiments the medium being supplemented with one or a combination of FN, laminin, VTN, ORM1, serpin, collagen, HP, TGM2, A2M, TNC, and/or coagulation factor comprises dexamethasone at a concentration of 10-100 nM, at a concentration of 20-90 nM, at a concentration of 30-80 nM, at a concentration of 40-70 nM at a concentration of 50 μg/mL.

In some embodiments the medium being supplemented with one or a combination of FN, laminin, VTN, ORM1, serpin, collagen, HP, TGM2, A2M, TNC, and/or coagulation factor comprises EGF at a concentration of 0.0-10 ng/mL, at a concentration of 1-9 ng/mL, at a concentration of 2-9 ng/mL, at a concentration of 3-8 ng/mL, at a concentration of 4-7 ng/mL, at a concentration of 5 ng/mL, at a concentration of 0 ng/mL

In some embodiments the medium being supplemented with one or a combination of FN, laminin, VTN, ORM1, serpin, collagen, HP, TGM2, A2M, TNC, and/or coagulation factor comprises Asc-2P at a concentration of 0.01-1 mM, at a concentration of 0.02-0.8 mM, at a concentration of 0.04-0.6 mM, at a concentration of 0.06-0.4 mM, at a concentration of 0.08-0.2 mM, at a concentration of 0.1 mM.

In some embodiments the medium being supplemented with one or a combination of FN, laminin, VTN, ORM1, serpin, collagen, HP, TGM2, A2M, TNC, and/or coagulation factor comprises HEPES at a concentration of 10-50 mM, at a concentration of 13-40 mM, at a concentration of 16-30 mM, at a concentration of 18-25 mM, at a concentration of 20 mM.

In some embodiments the medium being supplemented with one or a combination of FN, laminin, VTN, ORM1, serpin, collagen, HP, TGM2, A2M, TNC, and/or coagulation factor comprises penicillin at a concentration of 10-300 IU/mL, at a concentration of 30-250 IU/mL, at a concentration of 50-200 IU/mL, at a concentration of 70-150 IU/mL, at a concentration of 100 IU/mL.

In some embodiments the medium being supplemented with one or a combination of FN, laminin, VTN, ORM1, serpin, collagen, HP, TGM2, A2M, TNC, and/or coagulation factor comprises streptomycin at a concentration of 10-300 μg/mL, at a concentration of 30-250 μg/mL, at a concentration of 50-200 μg/mL, at a concentration of 70-150 μg/mL, at a concentration of 100 μg/mL.

In some embodiments the medium being supplemented with one or a combination of FN, laminin, VTN, ORM1, serpin, collagen, HP, TGM2, A2M, TNC, and/or coagulation factor comprises heat-inactivated FBS or human serum at a concentration of 2-20%, at a concentration of 4-18%, at a concentration of 6-16%, at a concentration of 8-14%, at a concentration of 10%.

In some embodiments the medium is supplemented with all of the humoral factors, FN, laminin, VTN, ORM1, serpin, collagen, HP, TGM2, A2M, TNC, and/or coagulation factor. In some embodiments the medium is supplemented with a combination of some of the humoral factors, FN, laminin, VTN, ORM1, serpin, collagen, HP, TGM2, A2M, TNC, and/or coagulation factor. In some embodiments the medium is supplemented with at least one of the humoral factors, FN, laminin, VTN, ORM1, serpin, collagen, HP, TGM2, A2M, TNC, and/or coagulation factor.

In some embodiments, the FN is supplemented to the medium to a concentration of 1-10000 ng/mL.

In some embodiments, the laminin is supplemented to the medium to a concentration of 1-10000 μg/mL.

In some embodiments, the VTN is supplemented to the medium to a concentration of 0.1-1000 ng/mL.

In some embodiments, the ORM1 is supplemented to the medium to a concentration of 0.1-1000 ng/mL.

In some embodiments, the serpin is supplemented to the medium to a concentration of 0.1-1000 μg/mL.

In some embodiments, the collagen is supplemented to the medium to a concentration of 1-10000 ng/mL.

In some embodiments, the HP is supplemented to the medium to a concentration of 0.01-100 mg/mL.

In some embodiments, the TGM2 is supplemented to the medium to a concentration of 0.01-100 ng/mL.

In some embodiments, the A2M (alpha-2 macroglobulin) is supplemented to the medium to a concentration of 0.1-1000 μg/mL.

In some embodiments, the TNC (Tenascin C) is supplemented to the medium to a concentration of 0.01-100 ng/mL.

In some embodiments, the coagulation factor is supplemented to the medium to a concentration of 0.1-1000 mg/mL.

In some embodiments, the medium is supplemented with at least two of the following: fibronectin (FN), vitronectin (VTN), haptoglobin (HP), alpha-2 macroglobulin (A2M), fibrinogen, and Tenascin C (TNC). In some embodiments, the medium is supplemented with at least three of the following: fibronectin (FN), vitronectin (VTN), haptoglobin (HP), alpha-2 macroglobulin (A2M), fibrinogen, and Tenascin C (TNC). In some embodiments, the medium is supplemented with at least four of the following: fibronectin (FN), vitronectin (VTN), haptoglobin (HP), alpha-2 macroglobulin (A2M), fibrinogen, and Tenascin C (TNC). In some embodiments, the medium is supplemented with at least five of the following: fibronectin (FN), vitronectin (VTN), haptoglobin (HP), alpha-2 macroglobulin (A2M), fibrinogen, and Tenascin C (TNC). In some embodiments, the medium is supplemented with at least fibronectin (FN), vitronectin (VTN), haptoglobin (HP), alpha-2 macroglobulin (A2M), fibrinogen, and Tenascin C (TNC).

In some embodiments, the medium is supplemented with at least two of the following: Fibrinogen, Vitronectin, Fibronectin, Tenascin C, and Haptoglobin. In some embodiments, the medium is supplemented with at least three of the following: Fibrinogen, Vitronectin, Fibronectin, Tenascin C, and Haptoglobin. In some embodiments, the medium is supplemented with at least four of the following: Fibrinogen, Vitronectin, Fibronectin, Tenascin C, and Haptoglobin. In some embodiments, the medium is supplemented with at least five of the following: Fibrinogen, Vitronectin, Fibronectin, Tenascin C, and Haptoglobin. In some embodiments, the medium is supplemented with at least six of the following: Fibrinogen, Vitronectin, Fibronectin, Tenascin C, and Haptoglobin. In some embodiments, the medium is supplemented with at least two Fibrinogen, Vitronectin, Fibronectin, Tenascin C, and Haptoglobin.

In some aspects of the inventions, the medium supplemented with one or a combination of FN, laminin, VTN, ORM1, serpin, collagen, HP, TGM2, A2M, TNC, and/or coagulation factor is used for the rapid and robust recovery from the cell injury or stress associated with cell isolation procedures and freeze/thaw cycle(s).

In some aspects of the inventions, the medium supplemented with one or a combination of FN, laminin, VTN, ORM1, serpin, collagen, HP, TGM2, A2M, TNC, and/or coagulation factor is used to culture HH in vitro to exhibit a functionality at a comparable level to that of the human liver, thereby being suitable for a variety of experimental applications.

In some aspects of the inventions, the medium supplemented with one or a combination of FN, laminin, VTN, ORM1, serpin, collagen, HP, TGM2, A2M, TNC, and/or coagulation factor is used to assay performance of a candidate agent in promoting alcohol metabolism by HH, to promote xenobiotics metabolism by HH, or to assay toxicity of the candidate agent to HH.

In some aspects of the inventions, the medium supplemented with one or a combination of FN, laminin, VTN, ORM1, serpin, collagen, HP, TGM2, A2M, TNC, and/or coagulation factor is used to assay toxicity of an agent to human hepatocytes (HH) or drug metabolism and pharmacokinetics (DMPK) of the agent.

In some aspects of the assay method, the agent is an alcohol compound comprising ethanol, methanol, ethylene glycol, isopropanol, or a mixture thereof.

In some aspects of the assay method, the contacting and the measuring are performed in a tight-sealing apparatus.

In some aspects of the assay method, the level of toxicity is measured via quantifying expression or function of xenobiotics-metabolizing enzymes such as, but not limited to Cytochrome P450 (CYP) enzymes in the quantity of HH.

In some aspects of the assay method, the level of toxicity is measured via quantifying the toxic metabolites of alcohol such as acetaldehyde, or reduction of glutathione, the activation status of cell death pathways in the quantity of HH.

In various embodiments, the medium supplemented with one or a combination of FN, laminin, VTN, ORM1, serpin, collagen, HP, TGM2, A2M, TNC, and/or coagulation factor is used for liver cell biology research. The current major limitation in the research of liver cell biology is the lack of stable in vitro culture system of terminally differentiated HH. For example, cryopreserved and/or freshly isolated PHH is the mainstay tool that most pharmaceutical industry utilizes for the drug safety screening as well as drug metabolism and pharmacokinetics (DMPK) studies at the preclinical trial stage. The term “freshly isolated” refers to less than 168 hours after isolation of the hepatocytes. For example, freshly isolated hepatocytes (e.g. PHH) include unfrozen hepatocytes within 168 hours, within 72 hours, within 36 hours, within 24 hours, within 12 hours, or within 6 hours of isolation. In a preferred embodiment of the invention, the freshly isolated hepatocytes are unfrozen hepatocytes within 6 hours of isolation. The term “cryopreserved” refers to hepatocytes that have been frozen using, for example, liquid nitrogen, a programmed freezer, a deep freezer, and the like.

As such in various embodiments, liver cells are placed in contact with the medium supplemented with one or a combination of FN, laminin, VTN, ORM1, serpin, collagen, HP, TGM2, A2M, TNC, and/or coagulation factor, and are cultured in the medium.

An aspect of the invention provides a medium supplemented with one or a combination of FN, laminin, VTN, ORM1, serpin, collagen, HP, TGM2, A2M, TNC, and/or coagulation factor for use in cultivating terminally differentiated PHH, HLCM-HH, hepatocytes derived from another chimeric animal (e.g., rat, sheep, pig, monkey) with humanized liver, primary hepatocytes from another species, e.g., dog, mouse, rat, and monkey, and/or cryopreserved and/or freshly isolated PHH, HLCM-HH, hepatocytes derived from another chimeric animal (e.g., rat, sheep, pig, monkey) with humanized liver, and primary hepatocytes from another species, e.g., dog, mouse, rat, and monkey. As such in various embodiments, terminally differentiated PHH and HLCM-HH are placed in contact with the medium supplemented with one or a combination of FN, laminin, VTN, ORM1, serpin, collagen, HP, TGM2, A2M, TNC, and/or coagulation factor, and are cultured in the medium.

In some embodiments, the biological effect of a medium supplemented with one or a combination of FN, laminin, VTN, ORM1, serpin, collagen, HP, TGM2, A2M, TNC, and/or coagulation factor supports the function of nonparenchymal cells (NPC) of the liver, such as hepatic stellate cells (HSC), liver sinusoidal endothelial cells (LSEC), and hepatic macrophage (Kupffer cells). As such in various embodiments, nonparenchymal cells (NPC) of the liver, such as hepatic stellate cells (HSC), liver sinusoidal endothelial cells (LSEC), and hepatic macrophage (Kupffer cells) are placed in contact with the medium supplemented with one or a combination of FN, laminin, VTN, ORM1, serpin, collagen, HP, TGM2, A2M, TNC, and/or coagulation factor, and are cultured in the medium.

In some embodiments, the medium supplemented with one or a combination of FN, laminin, VTN, ORM1, serpin, collagen, HP, TGM2, A2M, TNC, and/or coagulation factor is used to prevent the dedifferentiation of terminally differentiated in vitro cultured HH, HLCM-HH, hepatocytes derived from another chimeric animal (e.g., rat, sheep, pig, monkey) with humanized liver, PHH, and and/or primary hepatocytes from another species, e.g., dog, mouse, rat, and monkey. As such in various embodiments, HH, HLCM-HH, hepatocytes derived from another chimeric animal with humanized liver, PHH, and and/or primary hepatocytes from another species are placed in contact with the medium supplemented with one or a combination of FN, laminin, VTN, ORM1, serpin, collagen, HP, TGM2, A2M, TNC, and/or coagulation factor, and are cultured in the medium.

A further aspect of the invention provides a medium supplemented with one or a combination of FN, laminin, VTN, ORM1, serpin, collagen, HP, TGM2, A2M, TNC, and/or coagulation factor for use in prohibiting the transdifferentiation of HH, HLCM-HH, hepatocytes derived from another chimeric animal (e.g., rat, sheep, pig, monkey) with humanized liver, PHH, and and/or primary hepatocytes from another species, e.g., dog, mouse, rat, and monkey. As such in various embodiments, HH, HLCM-HH, hepatocytes derived from another chimeric animal with humanized liver, PHH, and and/or primary hepatocytes from another species are placed in contact with the medium supplemented with one or a combination of FN, laminin, VTN, ORM1, serpin, collagen, HP, TGM2, A2M, TNC, and/or coagulation factor, and are cultured in the medium.

A further aspect of the invention provides a medium supplemented with one or a combination of FN, laminin, VTN, ORM1, serpin, collagen, HP, TGM2, A2M, TNC, and/or coagulation factor for use in promoting the recovery of cryopreserved and/or freshly isolated HH, HLCM-HH, hepatocytes derived from another chimeric animal (e.g., rat, sheep, pig, monkey) with humanized liver, PHH, and and/or primary hepatocytes from another species, e.g., dog, mouse, rat, and monkey. As such in various embodiments, cryopreserved and/or freshly isolated HH, HLCM-HH, hepatocytes derived from another chimeric animal with humanized liver, PHH, and and/or primary hepatocytes from another species are placed in contact with the medium supplemented with one or a combination of FN, laminin, VTN, ORM1, serpin, collagen, HP, TGM2, A2M, TNC, and/or coagulation factor, and are cultured in the medium.

A further aspect of the invention provides a medium supplemented with one or a combination of FN, laminin, VTN, ORM1, serpin, collagen, HP, TGM2, A2M, TNC, and/or coagulation factor for use in promoting the recovery of freshly isolated HH, HLCM-HH, hepatocytes derived from another chimeric animal (e.g., rat, sheep, pig, monkey) with humanized liver, PHH, and and/or primary hepatocytes from another species, e.g., dog, mouse, rat, and monkey from stresses/injuries associated with cell procurement processes. As such in various embodiments, cryopreserved and/or freshly isolated HH, HLCM-HH, hepatocytes derived from another chimeric animal with humanized liver, PHH, and and/or primary hepatocytes from another species are placed in contact with the medium supplemented with one or a combination of FN, laminin, VTN, ORM1, serpin, collagen, HP, TGM2, A2M, TNC, and/or coagulation factor, and are cultured in the medium.

A further aspect of the invention provides a medium supplemented with one or a combination of FN, laminin, VTN, ORM1, serpin, collagen, HP, TGM2, A2M, TNC, and/or coagulation factor for use in protecting HH, HLCM-HH, hepatocytes derived from another chimeric animal (e.g., rat, sheep, pig, monkey) with humanized liver, PHH, and and/or primary hepatocytes from another species, e.g., dog, mouse, rat, and monkey, from the cellular stress associated with the cryopreservation, which may further support the cell recovery upon thawing. As such in various embodiments, HH, HLCM-HH, hepatocytes derived from another chimeric animal with humanized liver, PHH, and and/or primary hepatocytes from another species are placed in contact with the medium supplemented with one or a combination of FN, laminin, VTN, ORM1, serpin, collagen, HP, TGM2, A2M, TNC, and/or coagulation factor, and are cultured in the medium.

A further aspect of the invention provides a medium supplemented with one or a combination of FN, laminin, VTN, ORM1, serpin, collagen, HP, TGM2, A2M, TNC, and/or coagulation factor for use in supporting the maintenance and function of NPC function and morphology. In some embodiments, a CMHH is provided for use in co-culturing with NPCs, and/or for use in suspending human hepatocytes in cryopreservation. As such in various embodiments, hepatocytes are placed in contact with the medium supplemented with one or a combination of FN, laminin, VTN, ORM1, serpin, collagen, HP, TGM2, A2M, TNC, and/or coagulation factor, and are cryopreserved in the medium.

A further aspect of the invention provides a medium supplemented with one or a combination of FN, laminin, VTN, ORM1, serpin, collagen, HP, TGM2, A2M, TNC, and/or coagulation factor for use in supporting/enhancing the functionality and longevity of HH, HLCM-HH, hepatocytes derived from another chimeric animal (e.g., rat, sheep, pig, monkey) with humanized liver, PHH, and and/or primary hepatocytes from another species, e.g., dog, mouse, rat, and monkey, in the format of spheroid, organoid, or 3D culture, as well as for the development of organ-chips format. As such in various embodiments, spheroid, organoid, or 3D culture of HH, HLCM-HH, or hepatocytes are placed in contact with the medium supplemented with one or a combination of FN, laminin, VTN, ORM1, serpin, collagen, HP, TGM2, A2M, TNC, and/or coagulation factor, and are cultured in the medium.

A further aspect of the invention provides a medium supplemented with one or a combination of FN, laminin, VTN, ORM1, serpin, collagen, HP, TGM2, A2M, TNC, and/or coagulation factor for use in enhancing the utility of HH, HLCM-HH, hepatocytes derived from another chimeric animal (e.g., rat, sheep, pig, monkey) with humanized liver, PHH, and and/or primary hepatocytes from another species, e.g., dog, mouse, rat, and monkey, for the production of HLCM.

A further aspect of the invention provides a medium supplemented with one or a combination of FN, laminin, VTN, ORM1, serpin, collagen, HP, TGM2, A2M, TNC, and/or coagulation factor for use in supporting the cell fate maintenance of HH, HLCM-HH, hepatocytes derived from another chimeric animal (e.g., rat, sheep, pig, monkey) with humanized liver, PHH, and and/or primary hepatocytes from another species, e.g., dog, mouse, rat, and monkey; thereby CMHH greatly enhances the utility of these cells for various types of liver cell biology studies, such as DMPK, drug screening, infectious diseases, and metabolic disease. As such in various embodiments, HH, HLCM-HH, hepatocytes derived from another chimeric animal with humanized liver, PHH, and/or primary hepatocytes from another species, are placed in contact with the medium supplemented with one or a combination of FN, laminin, VTN, ORM1, serpin, collagen, HP, TGM2, A2M, TNC, and/or coagulation factor, and are cultured in the medium in these studies.

In various embodiments, the medium supplemented with one or a combination of FN, laminin, VTN, ORM1, serpin, collagen, HP, TGM2, A2M, TNC, and/or coagulation factor is used for cell fate maintenance of PHH and HLCM-HH and HH derived from another chimeric animal with humanized liver; thereby the medium supplemented with one or a combination of EN, laminin, VTN, ORM1, serpin, collagen, HP, TGM2, A2M, TNC, and/or coagulation factor greatly enhances the utility of these cells for various types of liver cell biology studies, such as DMPK, drug screening, infectious diseases, and metabolic diseases.

In various embodiments, the medium supplemented with one or a combination of FN, laminin, VTN, ORM1, serpin, collagen, HP, TGM2, A2M, TNC, and/or coagulation factor is used in regenerative medicine and stem cell biology. medium supplemented with one or a combination of FN, laminin, VTN, ORM11, serpin, collagen, HP, TGM2, A2M, TNC, and/or coagulation factor can be an essential factor that enables the final stage of iHep maturation to the terminally differentiated human hepatocytes. In some embodiments, the medium supplemented with one or a combination of FN, laminin, VTN, ORM1, serpin, collagen, HP, TGM2, A2M, TNC, and/or coagulation factor is used in the preparation of a hepatocyte transplantation therapy, e.g., medium supplemented with one or a combination of FN, laminin, VTN, ORM1, serpin, collagen, HP, TGM2, A2M, TNC, and/or coagulation factor used in the mass production of iPS-cells derived matured human hepatocytes, which can be a substitution of LT

A further aspect of the invention provides a medium supplemented with one or a combination of FN, laminin, VTN, ORM1, serpin, collagen, HP, TGM2, A2M, TNC, and/or coagulation factor for use in facilitating the further maturation of iHep (stem cell derived hepatic bipotential progenitor cells) to terminally differentiated hepatocytes.

A further aspect of the invention provides a medium supplemented with one or a combination of FN, laminin, VTN, ORM1, serpin, collagen, HP, TGM2, A2M, TNC, and/or coagulation factor for use in preventing the differentiation of iHep (stem cell derived hepatic bipotential progenitor cells) to cholangiocytes or other cell types.

A further aspect of the invention provides a medium supplemented with one or a combination of FN, laminin, VTN, ORM1, serpin, collagen, HP, TGM2, A2M, TNC, and/or coagulation factor for use in promoting the re-differentiation of Chemically Induced Liver Progenitors (CLiPs) to the matured hepatocytes.

A further aspect of the invention provides a medium supplemented with one or a combination of FN, laminin, VTN, ORM1, serpin, collagen, HP, TGM2, A2M, TNC, and/or coagulation factor for use in enhancing hepatic functions of hepatoma cells (e.g., HepaRG cells, HepG2, and Huh7 cells).

In some embodiments, the medium supplemented with one or a combination of FN, laminin, VTN, ORM1, serpin, collagen, HP, TGM2, A2M, TNC, and/or coagulation factor is used to enhance hepatic functions of HepaRG.

A further aspect of the invention provides a medium supplemented with one or a combination of FN, laminin, VTN, ORM1, serpin, collagen, HP, TGM2, A2M, TNC, and/or coagulation factor for use in allowing for the expansion/propagation of the iHep and CLiPs in the liver of HLCM-host mouse. In some embodiments, the invention provides a medium supplemented with one or a combination of FN, laminin, VTN, ORM1, serpin, collagen, HP, TGM2, A2M, TNC, and/or coagulation factor for use in allowing for the expansion/propagation of the PHH, and/or primary hepatocytes from another species, e.g., dog, mouse, rat, and monkey, in the liver of HLCM-host mouse.

A further aspect of the invention provides a medium supplemented with one or a combination of FN, laminin, VTN, ORM1, serpin, collagen, HP, TGM2, A2M, TNC, and/or coagulation factor for use in promoting the regression of chronic liver disease/liver fibrosis, as a result of hepatocyte transplantation therapy.

A further aspect of the invention provides a medium supplemented with one or a combination of FN, laminin, VTN, ORM1, serpin, collagen, HP, TGM2, A2M, TNC, and/or coagulation factor for use in adjuvant therapy for liver cancer.

A further aspect of the invention provides a medium supplemented with one or a combination of FN, laminin, VTN, ORM1, serpin, collagen, HP, TGM2, A2M, TNC, and/or coagulation factor for use in supporting the differentiation and cell fate maintenance of iHep, CLiPs, and HepaRG, which allows the stable and long-term culture of these cells without compromising the characteristics of matured hepatocytes, therefore medium supplemented with one or a combination of FN, laminin, VTN, ORM1, serpin, collagen, HP, TGM2, A2M, TNC, and/or coagulation factor greatly enhances the utility of these cells for various types of liver cell biology studies, such as DMPK, drug screening, infectious diseases, and metabolic diseases.

A further aspect of the invention provides a medium supplemented with one or a combination of FN, laminin, VTN, ORM1, serpin, collagen, HP, TGM2, A2M, TNC, and/or coagulation factor for use in differentiating Chemically Induced Liver Progenitors (CLiP) cells.

In some embodiments, the medium supplemented with one or a combination of FN, laminin, VTN, ORM1, serpin, collagen, HP, TGM2, A2M, TNC, and/or coagulation factor is used to promote the re-differentiation of CLiPs to the matured hepatocytes.

In various embodiments, medium supplemented with one or a combination of FN, laminin, VTN, ORM1, serpin, collagen, HP, TGM2, A2M, TNC, and/or coagulation factor is used to cultivate and maintain hepatocytes that could include HH, PHH from other species, e.g., dog, mouse, rat, and monkey, hepatocytes derived from humanized liver chimeric mouse (HLCM), or hepatocytes derived from another chimeric animal with humanized liver (e.g., hepatocytes derived from rat, sheep, pig, or monkey).

In some embodiments, the medium supplemented with one or a combination of FN, laminin, VTN, ORM1, serpin, collagen, HP, TGM2, A2M, TNC, and/or coagulation factor is used for the hepatocytes transplantation (such as iHeps further differentiated by a medium supplemented with one or a combination of FN, laminin, VTN, ORM1, serpin, collagen, HP, TGM2, A2M, TNC, and/or coagulation factor) to patients who suffer from any type liver diseases.

In certain embodiments, the HH comprise humanized liver chimeric mice derived-human hepatocytes (HLCM-HH), primary human hepatocytes (PHH), hepatocytes derived from a chimeric animal with humanized liver, e.g., rat, mouse, sheep, pig, or monkey, or a combination thereof. In some embodiments, the HH are PHH. In some embodiments, the HH are HLCM-HH. In some embodiments, the HH are derived from a chimeric animal other than mouse with a humanized liver

In certain embodiments, the HLCM-HH are obtained from a liver derived from a mouse injected with PHH or with previously isolated HLCM-HH into the spleen. In some embodiments, the HH are obtained from the liver derived from another animal (e.g., rat, mouse, sheep, pig, monkey, etc.) that was injected with PHH or with previously isolated HH from a humanized liver chimeric animal into the spleen.

In some embodiments, the mouse has a human hepatocyte replacement index (indicated by histological examination or blood concentration of HH derived factors (e.g., human albumin and human alpha 1 antitrypsin) of at least 10% before the HLCM-HH are obtained; or the chimeric animal with a humanized liver has a human hepatocyte replacement index at least 10% before the HH are isolated or obtained.

In a further embodiment, the HLCM-HH, the PHH, the hepatocytes derived from another chimeric animal with humanized liver, or a combination of any two or all three, are cultured at a cell density from 0.5×10⁵/cm² to 5×10⁵/cm².

In a further embodiment, the HH do not comprise any of HepG2 cells, Huh7 cells and murine hepatocytes. In another embodiment, the HH comprise HLCM-HH, which may include some mouse hepatocytes and mouse non-parenchymal cells in additional to HH.

In a further embodiment, the HH comprise HLCM-HH, and the method further comprises obtaining the HLCM-HH before the culturing step, wherein the obtaining of the HLCM-HH comprises isolating hepatocytes derived from a liver of a mouse injected with PHH or with previously isolated HLCM-HH into a spleen of the mouse, thereby obtaining the HLCM-HH.

In a further embodiment, the HH derived from the liver of the mouse are isolated via collagenase perfusion of the liver of the mouse, and the mouse has a human hepatocyte replacement index at least 10% before the HH are isolated.

### EXAMPLES

### Example 1: Role of Hepatocyte-Derived Extracellular Matrix and Coagulation Factors for the Fate Restoration and Maintenance of Human Hepatocytes

In search of the mechanism of how CMHH exhibits fate restoring and protective effect on in vitro cultured HLCM-HH as well as PHH, the Applicants' series of biochemical and genetic studies identified several key factors contained in CMHH: fibronectin (FN), e.g., FN1; laminin, e.g., LAMC1 and LAMC2; vitronectin (VTN); Orosomucoid 1 (ORM1) or Alpha-1-Acid Glycoprotein, (AGP1); serpin, e.g., SERPINA3; collagen, e.g., collagen IV Alpha 1 Chain; Haptoglobin (HP); Transglutaminase 2 (TGM2); alpha-2 macroglobulin (A2M); Tenascin C (TNC); and coagulation factor, e.g., FGA, FGB, and FGG). Based on this assessment, the supplementation of these factors into dHCGM or dHMM either individually or in combination, especially for the latter case, greatly support the fate restoration and maintenance of in vitro cultured HLCM-HH as well as PHH.

Based on the fate restorative and protective effects of FN, laminin, VTN, ORM1, serpin, collagen, HP, TGM2, A2M, TNC, and/or coagulation factor on in vitro cultured HH, these factors, either individually or in combination, have a great potential as cell culture medium supplements. Below are the itemized utilities.

### Regenerative Medicine and Stem Cell Biology

These studies demonstrate that FN, laminin, VTN, ORM1, serpin, collagen, HP, TGM2, A2M, TNC, and/or coagulation factor have potent fate determination effect on in vitro cultured HH; thus, these elements are necessary for the maturation of iHep into terminally differentiated human hepatocytes. This breakthrough finding will lead to the paradigm shift towards the development of hepatocyte transplantation therapy as a substitution of LT by enabling the mass production of iPS-cells derived matured human hepatocytes. Below are critical key features of for this category.
1. These factors serve as critical ingredients of cell culture medium that facilitates the further maturation of iHep (hepatic bipotential progenitor cells) to terminally differentiated hepatocytes.
2. Cell culture medium containing these factors prevents the trans-differentiation of iHep (hepatic bipotential progenitor cells) to cholangiocytes.
3. Cell culture medium containing these factors promote the re-differentiation of Chemically Induced Liver Progenitors (CLiPs) to the matured hepatocytes.
4. Cell culture medium containing these factors enhance hepatic functions of HepaRG
5. These factors serve as critical ingredients of cell culture medium enables the expansion/propagation of iHep and CLiPs in the liver of HLCM-host mouse.
6. Cell culture medium containing these factors enables the hepatocytes transplantation (such as iHeps) to patients who suffer from any type of liver diseases.
7. Cell culture medium containing these factors promote the regression of chronic liver disease/liver fibrosis.
8. These factors serve as adjuvant therapy for liver cancer.

### Liver Cell Biology Research

The current major limitation in the research of liver cell biology is the lack of stable in vitro culture system of terminally differentiated human hepatocytes. The supplementation of hepatocyte-intrinsic humoral factors, FN, laminin, VTN, ORM1, serpin, collagen, HP, TGM2, A2M, TNC, and/or coagulation factor, in the cell culture medium of human hepatocytes numerous advantages over current standard human hepatocytes culture methods. Please note that cryopreserved and/or isolated PHH is the mainstay tool that most pharmaceutical industry utilizes for the drug safety screening as well as DMPK studies at the preclinical trial stage. As the supplementation of these humoral factors, either individually or in combination, enables the complete fate restoration of cryopreserved and/or isolated PHH and also supports the maintenance of the characteristics of matured hepatocytes, this innovation has a tremendous business potential. In addition, the biological effect of these humoral factors appears to support the function and fate of nonparenchymal cells (NPC) of the liver, such as hepatic stellate cells (HSC), liver sinusoidal endothelial cells (LSEC), and hepatic macrophage (Kupffer cells).
1. These factors prevent the dedifferentiation of terminally differentiated in vitro cultured human hepatocytes, thereby maintaining the cell fate.
2. Cell culture medium containing these factors prohibits the transdifferentiation of in vitro cultured human hepatocytes into cholangiocytes-like cells.
3. These factors promote the recovery of cryopreserved and/or freshly isolated human hepatocytes to establish the stable in vitro culture.
4. Cell culture medium containing these factors protect PHH or HLCM-HH from the cellular stress during the cryopreservation, which may further support the cell recovery upon thawing.
5. Cell culture medium containing these factors support the maintenance of NPC function and morphology.
6. These factors promote support/enhance the functionality and longevity of HH or HLCM-HH in the format of spheroid, organoid, or 3D culture, as well as for the development of organ-chips format.

These factors promote enhance the utility of PHH or HLCM-HH for the production of HLCM.

### Uniqueness and Novelty of Hepatocyte-Intrinsic Humoral Factor Supplementation

The biological effect (hepatocyte fate restoration and maintenance) of hepatocyte-intrinsic humoral factors is robust and thus their supplementation into hepatocyte culture medium greatly enhances the utility of in vitro cultured hepatocytes, iHeps, CLiPs, and NPC of the liver. In brief, the production of one HLCM requires ~10⁵ PHH or HLCM-HH, which will be injected to the spleen of the host mouse (such as uPA-SCID strain). This results in the robust proliferation of PHH or HLCM-HH in the liver of host mouse in which endogenous murine hepatocytes with toxic transgene (uPA) overexpression diminish as a result of the competition with the transplanted human hepatocytes. After 8 weeks post-cell transplant, the liver of the host mouse will be replaced by human hepatocytes up to 95%. Consequently, the liver of one HLCM offers 2-3×10⁸ human hepatocytes, thereby enabling the mass production of HLCM-HH. Thus, this technical expertise provides the stable supply of HLCM-HH that enables the mass production of HLCM-HH. In addition, HLCM-HH exhibits greater viability and platability compared to PHH (for details, please refer to the review article: PMID: 32074631); hence, it preserves the genuine characteristics of terminally differentiated human hepatocytes during in vitro culture. This technical expertise led to the discovery that the culture medium of HLCM-HH (hereafter condition medium of human hepatocytes: CMHH) contains hepatocyte-intrinsic humoral factors that are necessary for the fate restoration and maintenance of human hepatocytes.

As described herein the Applicants delineate the key ingratiates that mediates the bioactivity of CMHH, which led to the identification of hepatocyte-intrinsic humoral factors, FN, laminin, VTN, ORM1, serpin, collagen, HP, TGM2, A2M, TNC, and/or coagulation factor, as critical supplements for the culture medium of human hepatocytes. There have been a few methodologies proposed to enhance the maturation and function of in vitro cultured hepatocytes such as Matrigel and five chemicals combination (namely, 5C) (PMID: 31023926). While there is some overlap between the biological effects of these methodologies and those of FN, laminin, VTN, ORM1, serpin, collagen, HP, TGM2, A2M, TNC, and/or coagulation factor, these factors, especially in combination, have more robust and physiological effects than Matrigel or 5C-containing medium. In addition, comprehensive transcriptome analysis reveals that the biological effect of hepatocyte-intrinsic humoral factors (FN, laminin, VTN, ORM1, serpin, collagen, HP, TGM2, A2M, TNC, and/or coagulation factor) is highly distinct from that of Matrigel or 5C.

### Material and Method

Humanized liver chimeric mice (HLCM) are first subjected to the quality assessment of the degree of human hepatocytes proliferation in the liver of the host mouse, namely replacement index (R.I.). The quality control of HLCM could be carried out through the assessment of serum/blood human albumin level, which has been widely accepted as a surrogate of the overall R.I. The Applicants primarily utilize HLCM with the blood albumin level higher than 10 mg/ml, corresponding to a RI greater than 70%. The HLCM are then subjected to the isolation of human hepatocytes (HH) via a collagenase perfusion-based method, followed by cell quality assessment (yield and viability). HLCM-derived human hepatocyte (HLCM-HH) are plated into a collagen-coated cell culture flask/dish at the cell density ranging 2.1-4.2×10′ cell/cm². Similarly, primary human hepatocytes (PHH), or HepaRG cells are plated into collagen coated cell culture flask/dish. These cells are cultured with dHCGM, dHMM (see appendix) or similar/equivalent (such as but not limited to dHCGM or dHMM containing DMSO2) for up to 21 days. The culture medium is replaced every 24-48 hours and the medium incubated with the cells is harvested and stored as conditioned medium of human hepatocytes (CMHH). The biological activity of CMHH in cell fate maintenance of terminally differentiated hepatocytes are evaluated via multi-readout assesment such as light microscopy for the morphology, immunofluorescent microscopy for the polarity, western blotting, and RT-qPCR for the expression abundance of hepatocyte marker genes. The CMHH exhibit the potent effect in supporting the fate restoration and maintenance of human hepatocytes; therefore, key substances are identified that mediates the biological effect of CMHH. First, the CMHH are filtered through various types of size exclusion columns to separate the biomolecules by molecular weight and their bioactivity assessment via aforementioned multi-readout approach point to a role of protein molecules. The follow up protein analysis revels that CMHH abundantly contains both serum proteins and extracellular matrixes. Next, the significance of each molecule is tested individually as well as in combination to further delineate the critical determinant of CMHH bioactivity, which led to the identification of fibronectin, laminin, and fibrinogen as the key ingredients that mediates the fate restoration and maintenance of human hepatocytes. Moreover, analysis of decellularized liver tissue of HLCM suggests that these molecules form macromolecular complex that serves as the bioscaffold for human hepatocytes. Lastly, in vitro studies addressing mechanism of the macromolecular complex formation with series of specific inhibitors determined that the activation of coagulation factor cascade plays a critical role in the multimerization of fibronectin, laminin, and fibrinogen

### APPENDIX

TABLE 1
Composition of dHCGM/d2HCGM Cell Culture Medium

| | | | | Final Conc. | |
|---|---|---|---|---|---|
| 1. | DMEM10 or a similar or equivalent | 500 | mL | | |
| | thereof | | | | |
| 2. | L-Proline Stock Solution (30 mg/mL) | 250 | μL | 15 | μg/mL |
| 3. | Insulin Stock Solution (10 mg/ml) | 12.5 | μL | 0.25 | μg/mL |
| 4. | Dexamethasone (Dex) Stock Solution | 2.5 | μL | 50 | nM |
| | (10 mM) | | | | |
| 5. | EGF Stock Solution (10 ug/mL) | 250 | μL | 5 | ng/mL |
| 6. | L-ascorbic Acid 2-Phosphate | 500 | μL | 0.1 | mM |

| | | | |
|---|---|---|---|
| | (Asc-2P) Stock Solution (0.1M) | | |
| 7. | DMSO or DMSO2 or a similar or | 10.2 mL or | 2% (DMSO) |
| | equivalent thereof | 6.38 g, | or 140.8 mM |
| | | respectively | (DMSO2) |

TABLE 2
Composition of dHMM/d2HMM Cell Culture Medium

| | | | | Final Conc. | |
|---|---|---|---|---|---|
| 1. | DMEM10 or a similar or equivalent | 500 | mL | | |
| | thereof | | | | |
| 2. | L-Proline Stock Solution (30 mg/mL) | 250 | μL | 15 | μg/mL |
| 3. | Insulin Stock Solution (10 mg/ml) | 12.5 | μL | 0.25 | μg/mL |
| 4. | Dexamethasone (Dex) Stock Solution | 2.5 | μL | 50 | nM |
| | (10 mM) | | | | |
| 5. | L-ascorbic Acid 2-Phosphate | 500 | μL | 0.1 | mM |

| | | | |
|---|---|---|---|
| | (Asc-2P) Stock Solution (0.1M) | | |
| 6. | DMSO or DMSO2 or a similar or | 10.2 mL or | 2% (DMSO) |
| | equivalent thereof | 6.38 g, | or 140.8 mM |
| | | respectively | (DMSO2) |

TABLE 3
DMEM10 Media

| | | Volume. | | Final Conc. |
|---|---|---|---|---|
| 1. | DMEM (without phenol red) | 450 | mL | |
| 2. | HEPES 1M solution | 10 | mL | 20 mM |
| 3. | Penicillin-Streptomycin | 5 | mL | 100 IU/mL, |
| | | | | 100 μg/mL |
| 4. | Heat inactivated*¹ fetal bovine | 50 | mL | 10% |

serum (FBS) Stock Solution*² or
Human serum

*¹After thawing FBS, heat it for 30 min at 56° C. (to deactivate the complement)
*²Added when used. Applicants found that without FBS, or with human serum, it could result in a comparable or even better biological effect.

### Example 2

### Materials and Methods

Isolation and Culture of Human Hepatocytes from Humanized Liver Chimeric Mice (HLCM-HHs)

As described in our previous publication (Tateno 2015), humanized liver chimeric mice (HLCM) were generated through xenotransplantation of cryopreserved or freshly isolated primary human hepatocytes (Lot:JFC, BioIVT, NY, US) into severe complex immune deficient (SCID) mice carrying a albumin enhancer/promoter-driven transgene (cDNA), urokinase type plasminogen activator, namely cDNA-uPA Tg/SCID). HLCM with blood human albumin level higher than 10 mg/mL were used for human hepatocyte isolation with two-step collagenase perfusion. The freshly isolated HLCM derived human hepatocytes (HLCM-HH) were plated on type I collagen coated culture dishes (BioCoat, Corning, NY, USA) at 1.05×10⁵ cells/cm². The plated HLCM-HHs were cultured with fresh dHCGM, INVITROGRO HI (HI, BioIVT) and Hepatocyte culture medium (HCM, Lonza, MD, USA), or these culture medium supplemented with the one or combination of the following factors, Fibrinogen (Cat #F4883-500M, Sigma-Aldrich, Inc., MO, USA) or Factor II inhibitor (F2i; Dabigatran etexilate, cat #SML2351-10 mg, Sigma-Aldrich, Inc., MO, USA), Factor X inhibitor (F10i; Rivoroxaban, Sigma-Aldrich), Vitronectin (VTN; Cat #2349-VN, R&D systems, MN, USA), Fibronectin (FN; Cat #4305-FNB, R&D systems), Tenascin C (TNC; Cat #3358-TC, R&D systems), Haptoglobin (HP; Cat #H3149-25KU, Sigma-Aldrich) for 7 days. For the preparation of HLCM-HH derived-conditioned medium, HLCM-HH were plated collagen coated T-75 flasks (BioCoat, Corning) at 2.1×10⁵ cells/cm². And culture supernatant was collected every two days from day 7 until day 21, and the collected culture supernatant were pooled and stored at −20° C. until use as conditioned medium.

### Gene Expression Analysis

Total RNA was extracted with Zymo-Quick RNA micro kit (Zymo Research, CA, USA) according to the provider's protocol. Total 1 g of RNA was used as a template for reverse transcription with Quanta cDNA synthesis super mix (Quantabio, MA, USA). Real-time quantitative PCR was performed with PowerUp SYBR Green Master Mix (ThermoFisher Scientific, MA, USA) and QuantStudio 5 systems (ThermoFisher Scientific) using human specific primer sets summarized in the Table 4. Relative gene expression levels were analyzed with the Delta-Dleta Ct method.

TABLE 4

| Gene | | |
|---|---|---|
| Symbol | Forward | Reverse |
| ANKRD1 | TGATGCGGTGAGACTGAA | CTGCCAGTGTAGCACCAGAT |
| | CC | |
| CYP2C9 | CCAGATCTGCAATAATTT | CAAGCTTTCAATAGTAAATT |
| | TTCTC | CAGATG |
| CYP2D6 | CTTGGACAAAGCCGTGA | GACAGCATTCAGCACCTC |
| KRT19 | TACAGCCACTACTACACGA | CCTGTTCCGTCTCAAACTTG |
| | CC | G |
| OATP1B1 | TCATACTCTGTGAAAACAA | CAGACTGGTTCCCATTGAC |
| | ATCAG | |
| TGFB1 | GCAGCACGTGGAGCTGTA | CAGCCGGTTGCTGAGGTA |
| TGFB2 | CCTTCTTCCCCTCCGAAAC | AGAGCACCTGGGACTGTCTG |

### Immunoblotting Analysis

Total 6 μL of fresh dHCGM and CM were mixed with Pro-Prep protein extraction solution (iNtRON Biotechnology Seongnam, South Korea) supplemented with protease inhibitor cocktail (MedChemExpress, NJ, USA). The mixtures were subjected to an SDS-PAGE followed by transfer to PVDF membranes for the detection of the proteins. Antibodies and its dilution factors were summarized in Table 5 (primary antibodies) and 6 (secondary antibodies).

TABLE 5
Primary antibodies

| | | Catalog | | | |
|---|---|---|---|---|---|
| Antigen | Vendor | Number | Clonality | Species | Concentration |
| ORM | Proteintech | 16439-1AP | Plolyclonal | Rabbit | 1:1000 |
| A2M | Proteintech | 66126-1-Ig | Monoclonal | Mouse | 1:1000 |
| COL4A1 | ABclonal | A10710 | Plolyclonal | Rabbit | 1:1000 |
| FGA | Proteintech | 20645-1-AP | Plolyclonal | Rabbit | 1:1000 |
| FN | Proteintech | 15613-1-AP | Plolyclonal | Rabbit | 1:1000 |
| HP | Proteintech | 66229-1-Ig | Monoclonal | Mouse | 1:1000 |
| LAMC1 | ABclonal | A16020 | Plolyclonal | Rabbit | 1:1000 |
| TGM2 | ABclonal | A0981 | Plolyclonal | Rabbit | 1:1000 |
| VTN | ABclonal | A13561 | Plolyclonal | Rabbit | 1:1000 |

TABLE 6
Secondary antibodies

| | | Catalog | |
|---|---|---|---|
| Antibody | Vendor | Number | Dilution |
| Peroxidase AffiniPure Goat | Jackson | 115-035-003 | 1:5000 |
| Anti-Mouse IgG (H + L) | ImmunoResearch | | |
| Peroxidase AffiniPure Goat | Jackson | 111-035-003 | 1:5000 |
| Anti-Rabbit IgG (H + L) | ImmunoResearch | | |

### Gene Knock-Down Analysis

For knock-down analysis of alpha 2 macroglobulin (A2M), negative control and A2M specific siRNA (Silencer select, Cat #; 4390824, assay ID; s819) were purchased from ThermoFisher scientific. The siRNAs were coated on a 96-well plate with transfection reagents and accelerator at CytoPathfinder (Tokyo, Japan). HLCM-HHs were plated on the transfection plates at 2.1×10⁵ cells/cm², and cultured for 9 days with dHCGM.

### Statistical Analysis

Statistical analysis was performed with GraphPad prism 9 (GraphPad Software, MA, USA). Significant differences were determined by Student's t-test. And Multiple comparisons were performed using one-way analysis or two-way analysis of variance followed by Dunnett's or Sidak's post hoc test.

### Results

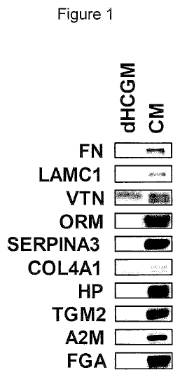

FIG. 1. Cultured HLCM-HHs secreted a variety of humoral factors into culture supernatant. Immunoblotting analysis was performed to identify humoral factors secreted by cultured HLCM-HHs using fresh dHCGM and CM. The results clearly indicated that CM contained all examined proteins, FN, LAMC1, VTN, ORM, SERPINA3, COL4A1, HP, TGM2, A2M FGA.

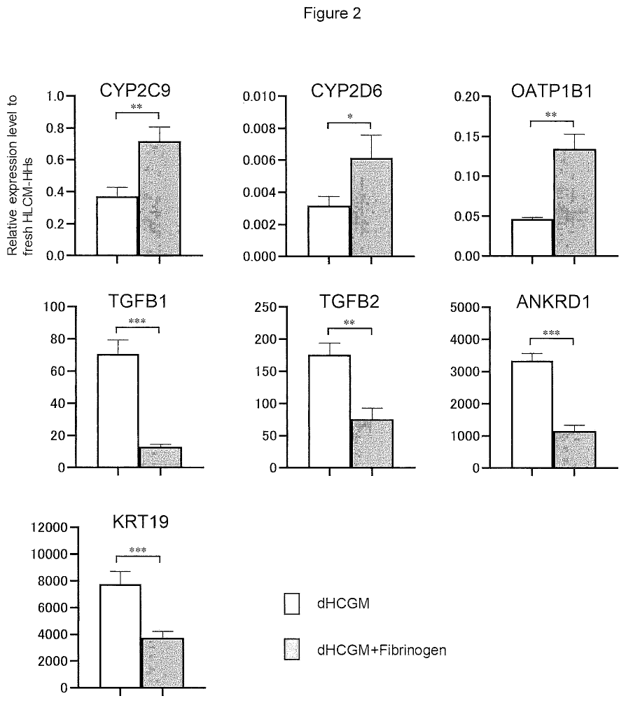

FIG. 2. Supplementation of fibrinogen to dHCGM enhances hepatic genes and prevent activation of cholangiocyte/liver progenitor cell marker expression.

Fibrinogen, one of the major components of HLCM-derived CM, was supplemented to fresh dHCGM (50 μg/mL) and examined the effects on expression of hepatic marker genes (CYP2C9, CYP2D6, OATP1B1) and cholangiocyte/liver progenitor marker genes (TGFB1, TGFB2, ANKRD1, KRT19). Results of qPCR clearly indicated that supplementation of fibrinogen could significantly enhance expression of the hepatic marker genes. On the other hand, expressions of cholangiocyte/liver progenitor cell markers were significantly inhibited in the HLCM-HHs cultured with dHCGM containing fibrinogen.

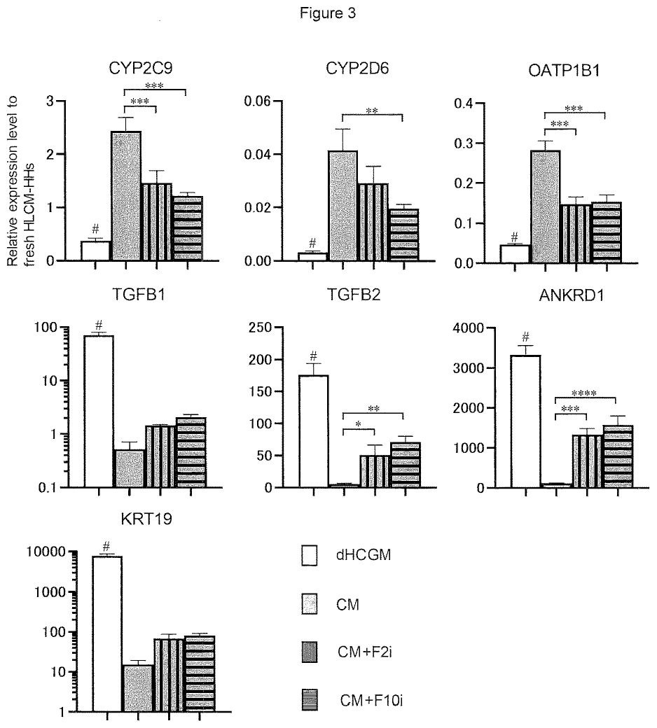

FIG. 3. Inhibition of coagulation cascade attenuated effects of CM on hepatocytes phenotypes. To examine the importance of activation of coagulation cascade for beneficial effects of CM, HLCM-HH were cultured with fresh dHCGM, CM, or CM containing F2i or F10i for 7 days and expression profiles of hepatocyte and cholangiocyte/liver progenitor cell markers were analyzed. Compare with FM-treated cells, CM-treated cells showed both higher expression of hepatocyte markers and lower expression of cholangiocyte/liver progenitor cell markers. However, supplementation of potent coagulation inhibitors to CM clearly attenuated the positive impacts of CM on maintenance of hepatocytes phenotypes. These results suggested that activation of coagulation cascade is one of the important pathways which essential to maximize the effects of CM on maintenance of hepatocytes functions in cultured HLCM-HHs.

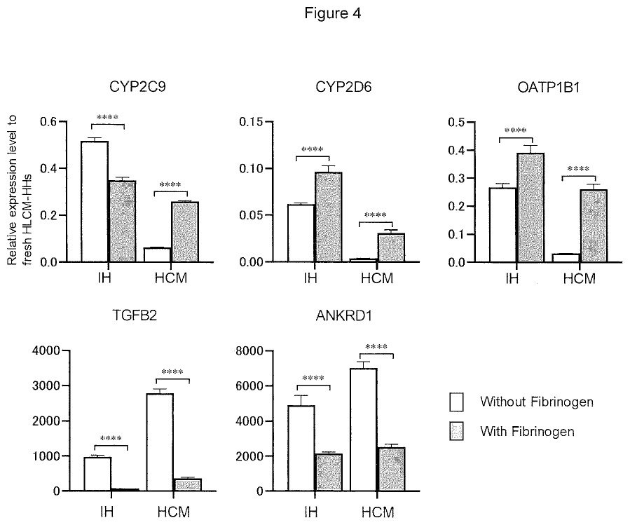

FIG. 4. Supplementation of fibrinogen could enhance hepatic phenotypes in HLCM-HHs cultured with commercially available hepatocytes culture medium.

To examine whether fibrinogen can enhance hepatic functions in human hepatocytes cultured with commercially available hepatocyte culture media, HLCM-HHs were cultured with two different hepatocyte culture media, INVITROGRO HI (IH) and Hepatocyte culture medium (HCM) in the presence or absence of fibrinogen supplementation. Expression of CYP2D6 and OATP1B1 were significantly activated by fibrinogen treatment. On the other hand, expression of cholangiocyte/liver progenitor cell markers, TGFB2 and ANKRD1, were significantly inhibited by fibrinogen treatment.

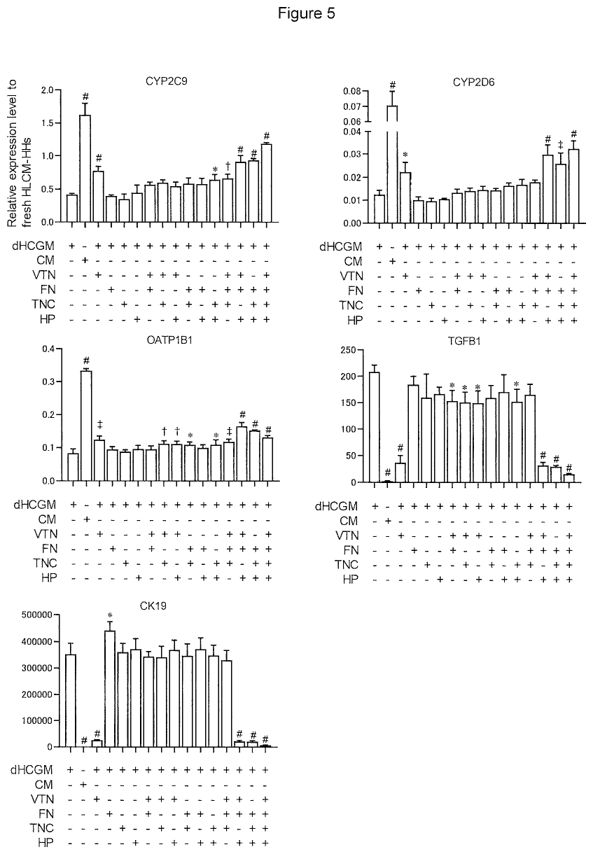

FIG. 5. Effect of the other CM components on the maintenance of hepatocytic gene expression profile.

Conditioned medium contains not only fibrinogen and coagulation factors, but also other liver matrisome components. We examined whether these matrisome components could also show positive impact on hepatocyte gene expression profile in cultured HHs. HLCM-HHs were cultured with fresh dHCGM (FM), CM, or FM supplemented with one or combination of the following liver matrisome components (VTN, FN, TNC, HP) included in CM. Results of gene expression analysis by qPCR indicated that tested liver matrisome components or combination of them at least partially mimicked the effect of CM on the hepatocyte marker genes and cholangiocyte/liver progenitor cell markers.

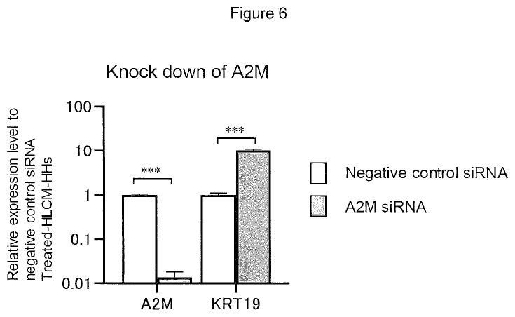

FIG. 6. Knock-down of alpha-2 macroglobulin (A2M) enhanced expression cholangiocyte marker expression in culture HLCM-HHs.

To examine importance of A2M, another conditioned medium component, A2M was knocked down by transfection of A2M specific siRNA. At 9 days after transfection, expression of A2M was dramatically suppressed to less than 5% of negative control siRNA transfected HLCM-HHs. Beside, KRT19, one of the cholangiocyte/liver progenitor cell markers, was significantly up-regulated in the A2M knocked-down HLCM-HHs. This result suggested that A2M, a component of CM, plays important role in the maintenance of hepatocyte phenotypes in the cultured human hepatocytes.

1.-32. (canceled)

33. A hepatocyte culture medium supplemented with at least one factor selected from the group consisting of:
Fibronectin (FN), laminin, vitronectin (VTN), orosomucoid 1 (ORM1), serpin, collagen, haptoglobin (HP), transglutaminase 2 (TGM2), alpha-2 macroglobulin (A2M), coagulation factors, Tenascin C (TNC), and combinations thereof.

34. The culture medium of claim 33, wherein the medium is a hepatocyte clonal growth medium (HCGM) or hepatocyte maintenance medium (HMM).

35. The culture medium of claim 33, wherein the laminin is laminin subunit gamma 1 (LAMC1), laminin subunit gamma 2 (LAMC2), or laminin subunit gamma 3 (LAMC3).

36. The culture medium of claim 33, wherein the coagulation factor is fibrinogen alpha chain (FGA), fibrinogen beta chain (FGB), or fibrinogen gamma chain (FGG).

37. The culture medium of claim 33, wherein the medium is further supplemented with DMSO or DMSO2.

38. The culture medium of claim 34, wherein the HCGM comprises, consists, or consists essentially of, Dulbecco's Modified Eagle's Medium (DMEM), L-proline, insulin, dexamethasone, EGF, and L-ascorbic acid 2-phosphate (Asc-2P).

39. The culture medium of claim 38, wherein the DMEM is DMEM-10, and the DMEM-10 comprises, consists, or consists essentially of, DMEM, HEPES, penicillin-streptomycin, and fetal bovine serum (FBS).

40. The culture medium of claim 38, wherein:
the L-proline is 15 μg/mL in the HCGM, the insulin is 0.25 μg/mL in the HCGM, the dexamethasone is 50 nM in the HCGM, the EGF is 5 ng/mL in the HCGM, the Asc-2P is 0.1 mM in the HCGM, the DMSO or the DMSO2 is 2% or 140.8 mM respectively in the HCGM, the HEPES is 20 mM in the HCGM, the penicillin is 100 IU/mL in the HCGM, the streptomycin is 100 μg/mL in the HCGM, and the FBS is heat-inactivated FBS at 10% in the HCGM; or
the L-proline is 5-25 μg/mL in the HCGM, the insulin is 0.1-0.5 μg/mL in the HCGM, the dexamethasone is 10-100 nM in the HCGM, the EGF is 1-10 ng/mL in the HCGM, the Asc-2P is 0.01-1 mM in the HCGM, the DMSO or the DMSO2 is 0.5%-5% 100-180 mM, respectively in the HCGM, the HEPES is 10-50 mM in the HCGM, the penicillin is 10-300 IU/mL in the HCGM, the streptomycin is 10-300 μg/mL in the HCGM, and the FBS is heat-inactivated FBS at 2-20% in the HCGM.

41. The culture medium of claim 34, wherein the HMM comprises, consists, or consists essentially of, Dulbecco's Modified Eagle's Medium (DMEM), L-proline, insulin, dexamethasone, and L-ascorbic acid 2-phosphate (Asc-2P).

42. The culture medium of claim 41, wherein the DMEM is DMEM-10, and the DMEM-10 comprises, consists, or consists essentially of, DMEM, HEPES, penicillin-streptomycin, and fetal bovine serum (FBS).

43. The culture medium of claim 41, wherein:
the L-proline is 15 μg/mL in the HMM, the insulin is 0.25 μg/mL in the HMM, the dexamethasone is 50 nM in the HMM, the Asc-2P is 0.1 mM in the HMM, the DMSO or the DMSO2 is 2% or 140.8 mM respectively in the HMM, the HEPES is 20 mM in the HMM, the penicillin is 100 IU/mL in the HMM, the streptomycin is 100 μg/mL in the HMM, and the FBS is heat-inactivated FBS at 10% in the HMM; or
the L-proline is 5-25 μg/mL in the HMM, the insulin is 0.1-0.5 μg/mL in the HMM, the dexamethasone is 10-100 nM in the HMM, the Asc-2P is 0.01-1 mM in the HMM, the DMSO or the DMSO2 is 0.5%-5% or 100-180 mM, respectively in the HMM, the HEPES is 10-50 mM in the HMM, the penicillin is 10-300 IU/mL in the HMM, the streptomycin is 10-300 μg/mL in the HMM, and the FBS is heat-inactivated FBS at 2-20% in the HMM.

44. The culture medium of claim 34, wherein:
the FN is supplemented to 10000 ng/mL in the HCGM or HMM;
the laminin is supplemented to 1-10000 μg/mL in the HCGM or HMM;
the VTN is supplemented to 0.1-1000 ng/mL in the HCGM or HMM;
the ORM1 is supplemented to 1-10000 mg/mL in the HCGM or HMM;
the serpin is supplemented to 0.1-1000 mg/mL in the HCGM or HMM;
the collagen is supplemented to 1-10000 ng/mL in the HCGM or HMM;
the HP is supplemented to 0.01-100 mg/mL in the HCGM or HMM;
the TGM2 is supplemented to 0.01-100 ng/mL in the HCGM or HMM;
the A2M is supplemented to 0.1-1000 mg/mL in the HCGM or HMM;
the coagulation factors is supplemented to 0.1-1000 mg/mL in the HCGM or HMM; and/or
the TNC is supplemented to 0.01-100 ng/mL in the HCGM or HMM.

45. A culture medium of claim 33 for use in in cultivating terminally differentiated human hepatocytes, hepatocytes derived from a chimeric animal with humanized liver, and/or cryopreserved and/or freshly isolated primary human hepatocytes.

46. A culture medium of claim 33 for use in differentiating hepatic bipotential progenitor cells (iHep) and/or chemically induced liver progenitors into terminally differentiated hepatocytes or matured hepatocytes, respectively.

47. A culture medium of claim 33 for use in co-culturing with nonparenchymal cells, and/or for use in suspending human hepatocytes in cryopreservation.

48. A culture medium of claim 33 for use in the expansion and/or propagation of iHep and Chemically Induced Liver Progenitors (CLiPs) in the liver of HLCM-host mouse.

49. A method of cultivating terminally differentiated human hepatocytes, hepatocytes derived from a chimeric animal with humanized liver, and/or cryopreserved and/or freshly isolated primary human hepatocytes, the method comprising culturing a quantity of hepatocytes in a cell culture medium of claim 33.

50. A method of differentiating hepatic bipotential progenitor cells (iHep) and/or chemically induced liver progenitors into terminally differentiated hepatocytes or matured hepatocytes, respectively, the method comprising culturing a quantity of hepatocytes in a cell culture medium of claim 33.

51. A method of co-culturing human hepatocytes with nonparenchymal cells, and/or suspending human hepatocytes in cryopreservation, the method comprising culturing a quantity of hepatocytes in a cell culture medium of claim 33.

52. A method of expanding and/or propagating iHep and CLiPs in the liver of HLCM-host mouse, the method comprising culturing a quantity of hepatocytes in a cell culture medium of claim 33.


SEQUENCE LISTING (14 sequences)

SEQ ID NO: 1 - DNA, 20 bp, synthetic construct
  source 1..20: mol_type = other DNA
  misc_feature 1..20: note = ANKRD1 Forward
tgatgcggtgagactgaacc

SEQ ID NO: 2 - DNA, 20 bp, synthetic construct
  source 1..20: mol_type = other DNA
  misc_feature 1..20: note = ANKRD1 Reverse
ctgccagtgtagcaccagat

SEQ ID NO: 3 - DNA, 23 bp, synthetic construct
  source 1..23: mol_type = other DNA
  misc_feature 1..23: note = CYP2C9 Forward
ccagatctgcaataatttttctc

SEQ ID NO: 4 - DNA, 26 bp, synthetic construct
  source 1..26: mol_type = other DNA
  misc_feature 1..26: note = CYP2C9 Reverse
caagctttcaatagtaaattcagatg

SEQ ID NO: 5 - DNA, 17 bp, synthetic construct
  source 1..17: mol_type = other DNA
  misc_feature 1..17: note = CYP2D6 Forward
cttggacaaagccgtga

SEQ ID NO: 6 - DNA, 18 bp, synthetic construct
  source 1..18: mol_type = other DNA
  misc_feature 1..18: note = CYP2D6 Reverse
gacagcattcagcacctc

SEQ ID NO: 7 - DNA, 21 bp, synthetic construct
  source 1..21: mol_type = other DNA
  misc_feature 1..21: note = KRT19 Forward
tacagccactactacacgacc

SEQ ID NO: 8 - DNA, 21 bp, synthetic construct
  source 1..21: mol_type = other DNA
  misc_feature 1..21: note = KRT19 Reverse
cctgttccgtctcaaacttgg

SEQ ID NO: 9 - DNA, 24 bp, synthetic construct
  source 1..24: mol_type = other DNA
  misc_feature 1..24: note = OATP1B1 Forward
tcatactctgtgaaaacaaatcag

SEQ ID NO: 10 - DNA, 19 bp, synthetic construct
  source 1..19: mol_type = other DNA
  misc_feature 1..19: note = OATP1B1 Reverse
cagactggttcccattgac

SEQ ID NO: 11 - DNA, 18 bp, synthetic construct
  source 1..18: mol_type = other DNA
  misc_feature 1..18: note = TGFB1 Forward
gcagcacgtggagctgta

SEQ ID NO: 12 - DNA, 18 bp, synthetic construct
  source 1..18: mol_type = other DNA
  misc_feature 1..18: note = TGFB1 Reverse
cagccggttgctgaggta

SEQ ID NO: 13 - DNA, 19 bp, synthetic construct
  source 1..19: mol_type = other DNA
  misc_feature 1..19: note = TGFB2 Forward
ccttcttcccctccgaaac

SEQ ID NO: 14 - DNA, 20 bp, synthetic construct
  source 1..20: mol_type = other DNA
  misc_feature 1..20: note = TGFB2 Reverse
agagcacctgggactgtctg